(19) United States
(12) Patent Application Publication
(10) Pub. No.: US 2009/0107509 A1
(43) Pub. Date: Apr. 30, 2009

(54) System for providing an open airway and methods of use thereof

(76) Inventor: Joseph A. Sniadach, Baltimore, MD (US)

Correspondence Address: J. Bruce Hoofnagle, P.O. Box 370, Lisbon, MD, 21765-0370, US

(21) Appl. No.: 11/980,855

(22) Filed: Oct. 31, 2007

(51) Int. Cl. A61M 16/04

(52) U.S. Cl. 128/207.14; 128/207.29

(57) ABSTRACT

In a system for dislodging a foreign body 60 from the vocal cords 52 of a patient 32, a retrograde intubation tube 64 is passed through the cricoidthyroid membrane 58, and between the vocal cords and the foreign body, and out of the oral cavity opening 34. A guide wire 84 is passed through, and extends outward from opposite ends 68 and 70 of, the tube 64. The carrier device 88, having a foreign-body dislodging means 104, is formed with a hollow opening 93, and is moved over, and guided by, the guidewire 84 and the tube 64 to a location adjacent the foreign body 60. The tube 64 and the guidewire 84 are removed, and the dislodging means 104 is manipulated to dislodge, and facilitate removal of, the foreign body 60.

### BACKGROUND OF THE INVENTION

This invention relates to a system for providing an airway, and to methods of use thereof. In particular, this invention relates to a system for removing a foreign body, such a as a food particle, which is lodged in a trachea opening, such as, for example, a glottic opening or vocal cords, of a patient, and to methods of using the system to accomplish the dislodge the foreign body to facilitate the removal thereof.

Occasionally, a person's airway may become blocked when a foreign body, such as a food particle or a non-food object, becomes undesirably lodged in the person's trachea opening, which includes the glottic opening and the vocal cords. Typically, this lodgement occurs when the person is eating food, and a particle of the food, instead of passing normally into the esophagus, becomes lodged in the trachea opening. Such lodgement could also occur when a person swallows, intentionally or unintentionally, a non-food object, which becomes lodged in the trachea opening. When such lodgement occurs, the person's airway is blocked, and the person is unable to ventilate or oxygenate, i.e., the person is unable to breath.

Many patients die each year from a lack of an adequate supply of oxygen due to the lodgement, or obstruction, of a foreign body in their trachea opening. Current means for removing such an obstruction relies on the Heimlich maneuver, or on the use of obstruction instrumentation by trained medical personnel under direct vision using a laryngoscope and forceps. In a situation where the Heimlich maneuver fails, the patient would proceed to unconsciousness and subsequent death, due to a lack of oxygenation.

This emergency situation requires quick action where seconds count, and where survival may likely be accompanied by brain injury. In this situation, CPR is ineffective because the upper airway obstruction of the patient prevents oxygenation, and the chance that CPR would dislodge the foreign body remains unlikely.

A currently used, highly invasive technique requires the forming of a significant opening in the neck to allow the passing of a tube into and down the trachea for the purpose of ventilating the patient. In this manner, the obstruction of the foreign body is by-passed. However, the significant neck opening must be formed, and the foreign body remains undesirably lodged in the patient's normal airway.

Since an airway obstruction may occur at the trachea opening, or farther into the trachea, direct visualization with forceps may not provide relief. Passage of an endotracheal tube may wedge the foreign body farther into the trachea, making it more difficult, if not impossible, to remove the foreign body within the extremely limited amount of time to accomplish such removal.

In a hospital environment, a fiberoptic bronchoscope may be placed down the trachea to retrieve the foreign body by use of a grasper. However, such sophisticated instrumentation is not available for use in an emergency situation in a non-hospital environment.

Another rarely used technique involves the passage of a needle through the cricothyroid membrane, and into the trachea. A guidewire is then placed through the needle with the intention of entering the oral cavity, where the guidewire is secured. An endotracheal tube is then placed over the guidewire, where, hopefully, the tube traverses the tongue and the oropharynx, and resides at the location of the entrance of the needle, with subsequent passage into the trachea after the guidewire is removed. This technique is used to establish an airway in a patient with a difficult airway, and would not work if the airway is obstructed because the wire would not provide sufficient stiffness to traverse the airway obstruction. Furthermore, if the wire did remain in place next to the lodged foreign body, the coaxial placement of the endotracheal tube would not traverse the obstruction of the foreign body.

Many commercially available emergency tracheotomy and cricothyroid kits can be used to provide an opening in the trachea, wherein a tube is subsequently inserted. However, these kits do not provide for retrograde endotracheal tube insertion where an endotracheal tube is placed into the mouth and advanced to the trachea. In addition, the facilities of these kits are highly invasive in order to obtain a surgical airway or opening in the neck, whereby a significant risk exists for damage to the thyroid or injury to nearby arteries and blood vessels. The facilities of these kits allow only for the passage of a tube distally through the opening.

Thus, there is a need for a system, and methods of using the system, which will quickly facilitate the removal of a foreign body lodged in the trachea opening of a patient, and allow ventilation and oxygenation of the patient.

### SUMMARY OF THE INVENTION

Therefore, it is an object of this invention to provide a system, and methods of using the system, which will quickly facilitate the removal of a foreign body lodged in the trachea opening of a patient, and allow ventilation and oxygenation of the patient.

With this and other objects in mind, this invention contemplates a system for providing an open airway for a patient, where the patient has a neck, a cricoidthyroid membrane, a glottic opening at the vocal cords, a pharyngeal area, an oral cavity and an oral cavity opening. A retrograde device positionable through the cricoidthyroid membrane, the glottic opening at the vocal cords, the pharyngeal area, the oral cavity and at least to a location outside of the patient's oral cavity opening. A carrier device having an end guidable along the retrograde intubation device from the location outside of the patient's oral cavity opening, through the oral cavity opening, the oral cavity, the pharyngeal area, and at least into a position adjacent the glottic opening.

This invention further contemplates a method of providing an open airway for a patient, where the patient has a neck, a cricoidthyroid membrane, a trachea, vocal cords, a glottic opening at the vocal cords, a pharyngeal area, an oral cavity and an oral cavity opening. In the method a retrograde device is positioned within, and through, the cricoidthyroid membrane, the glottic opening at the vocal cords, the pharyngeal area, the oral cavity and at least to a location outside of the patient's oral cavity opening. A carrier device is then moved guidingly along the retrograde device from the location outside of the patient's oral cavity opening, through the oral cavity opening, the oral cavity, the pharyngeal area, and at least into a position adjacent the glottic opening.

Other objects, features and advantages of the present invention will become more fully apparent from the following detailed description of the preferred embodiment, the appended claims and the accompanying drawings.

### BRIEF DESCRIPTION OF THE DRAWINGS

In the accompanying drawings:

FIG. 1 is a representation of the head and neck of a patient showing some of the anatomical structure thereof, which relate to the description below;

FIG. 2 is a side view showing a first embodiment of a hollow malleable retrograde intubation device, forming a component of a system for providing an open airway, and having a uniform diameter from a proximal end to a bevelled distal end thereof, in accordance with certain principles of the invention;

FIG. 3 is a side view showing a second embodiment of a hollow malleable retrograde intubation device, forming a component of the above-noted system for providing an open airway, and having a uniform diameter from a proximal end to a sharp distal end thereof, with an intermediate outer miniature inflatable cuff, in accordance with certain principles of the invention;

FIG. 4 is a side view showing a third embodiment of a hollow malleable retrograde intubation device, forming a component of the above-noted system for providing an open airway, which is tapered from a proximal end to a bevelled distal end thereof, with the intermediate outer miniature inflatable cuff, and a guidewire located therethrough, each of which form a component of the above-noted system for providing an open airway, in accordance with certain principles of the invention;

FIG. 5 is a side view showing a conventional inflatable cuff and the a comparatively smaller miniature inflatable cuff of FIGS. 3 and 4, which can be used with any of the respective embodiments of the retrograde intubation device of FIGS. 2, 3 and 4, in accordance with certain principles of the invention;

FIG. 6 is a partial section view showing a carrier device, forming a component of the system for providing an open airway, including an outer sleeve, an inner sleeve in telescoping assembly with the outer sleeve, and a foreign-body dislodging means attached to the inner sleeve, in accordance with certain principles of the invention; and

FIG. 7 is a top view showing the inner sleeve of the carrier device of FIG. 6, with spring-biased flexible fingers of the foreign-body dislodging means of FIG. 6 attached to the inner sleeve, in accordance with certain principles of the invention;

FIG. 8 is a partial side view showing the flexible fingers of FIG. 7, attached to the a distal end of the inner sleeve of FIG. 6, in an outwardly biased, extended position, in accordance with certain principles of the invention;

FIG. 9 is a top view showing the inner sleeve of the carrier device of FIG. 6, with an inflatable bowl-shaped cuff of the foreign-body dislodging means of FIG. 6 attached to the inner sleeve, in accordance with certain principles of the invention;

FIG. 10 is a side view showing the inflatable bowl-shaped cuff of FIG. 9 inflated and attached to the distal end of the inner sleeve of FIG. 6, in accordance with certain principles of the invention;

FIG. 11 is a side view showing the bevelled end of the retrograde intubation device of FIG. 2 in position for insertion into the cricoidthyroid membrane of the patient, and with a foreign body lodged within the inner space of the vocal cords at the glottic opening of the patient, in accordance with certain principles of the invention;

FIG. 12 is a side view showing a leading portion of the retrograde intubation device of FIG. 11 having been moved through the cricoidthyroid membrane, between the vocal cords and the foreign body lodged therebetween, with the bevelled distal end located within the oral cavity, adjacent the tongue, of the patient, in accordance with certain principles of the invention;

FIG. 13 is a side view showing the retrograde intubation device of FIGS. 11 and 12 having been moved to the extent that the bevelled distal end of the device extends outward from the oral cavity opening, and the proximal end of the device extends outward from cricoidthyroid membrane, in accordance with certain principles of the invention;

FIG. 14 is a side view showing the guidewire of FIG. 4 having been inserted through a hollow opening of the retrograde intubation device of FIG. 13, with the guidewire extending outward from the proximal and distal ends of the device, in accordance with certain principles of the invention;

FIG. 15 is a side view showing the assembly of the retrograde intubation device and the guidewire of FIG. 14, with the carrier device of FIG. 6 being moved over the guidewire toward the oral cavity opening, in accordance with certain principles of the invention;

FIG. 16 is a side view showing that the retrograde intubation device has been removed and the carrier device has been moved over the guidewire, through the opening of, and into, the oral cavity and the pharyngeal area, with the distal end of the carrier device located adjacent the vocal cords and the lodged foreign body, in accordance with certain principles of the invention;

FIG. 17 is a side view of the retrograde intubation device with the bevelled distal end having been moved past, and between, the foreign body and the vocal cords, with the miniature inflated cuff of FIGS. 3 and 5 positioned on the trachea side of the foreign body, in accordance with certain principles of the invention;

FIG. 18 is a side view showing the retrograde intubation device of FIG. 17 having been moved sufficiently to move the miniature inflated cuff into dislodging engagement with the foreign body, in accordance with certain principles of the invention;

FIG. 19 is a side view of the carrier device in the position illustrated in FIG. 16, with the distal end of the device having dislodged the foreign body from between the vocal cords, and farther into the trachea, toward the lungs;

FIG. 20 is a side view showing the distal end of the inner sleeve of the carrier device having been moved between, and past, the foreign body and the vocal cords, with the foreign-body dislodging means of FIGS. 6 through 10 located farther into the trachea, in accordance with certain principles of the invention;

FIG. 21 is a partial enlarged view showing a bowl-shaped cuff embodiment of the foreign-body dislodging means of FIG. 20 being inflated, with the opening of the cuff facing the foreign body, in accordance with certain principles of the invention;

FIG. 22 is a side view showing a laryngoscope being located to facilitate external visual sighting of the lodged foreign body, and with the inner sleeve, and the inflatable bowl-shaped cuff of FIGS. 9 and 10, of the carrier device, positioned for movement into the oral cavity and the pharyngeal area for dislodgement and removal of the foreign body, in accordance with certain principals of the invention; and

FIG. 23 is a side view showing an endotracheal tube, with a deflated cuff at the distal end thereof, positioned over the guidewire of FIG. 14, for guiding the tube into place for ventilation of the patient, in accordance with certain principles of the invention.

### DETAILED DESCRIPTION OF THE PREFERRED EMBODIMENTS OF THE INVENTION

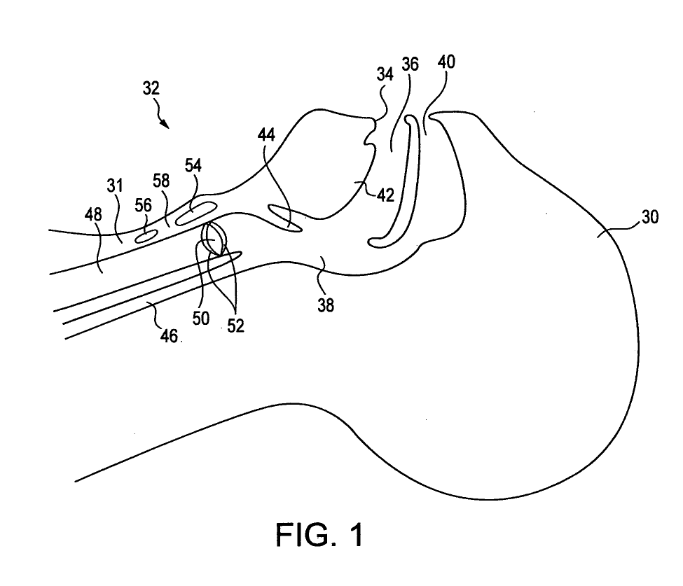

Referring to FIG. 1, a head 30 and a neck 31 of a patient 32 is illustrated to establish the anatomical structure in the area of the head and neck. The structure includes an oral cavity opening 34, an oral cavity 36, a pharyngeal area 38, and a nasal cavity 40. The structure further includes a tongue 42, an epiglottis 44, an esophagus 46, and a trachea 48. In addition, the structure includes a glottic opening 50 to the trachea 48, vocal cords 52, located at the glottic opening, thyroid cartilage 54, cricoid cartilage 56, and a cricoidthyroid membrane 58, located in a space between the thyroid and the cricoid cartilages.

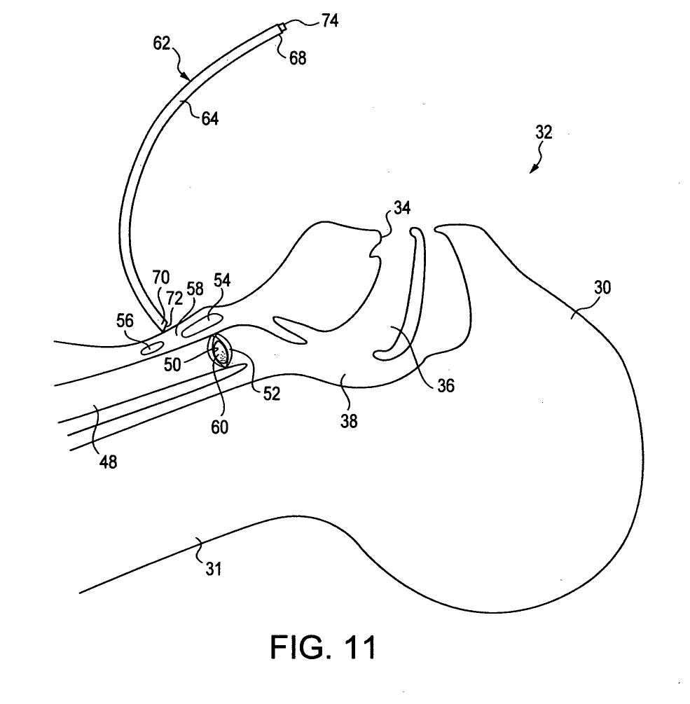

Occasionally, a foreign body 60 (FIG. 11) becomes undesirably lodged in the glottic opening 50, between the vocal cords 52, which results in the inability of the patient 32 to breath, and thereby ventilate and oxygenate. Without the ability to breath, the patient would quickly become unconscious, and death would quickly follow due to the lack of oxygenation. In a hospital environment, and with highly skilled and professional caregivers, various techniques and instrumentation could be employed in an attempt to remove the lodged foreign body 60.

However, many times, lodgement of the foreign body occurs in a non-hospital environment, where minimally trained caregivers are called upon to quickly attempt to dislodge the foreign body, or to provide ventilation and oxygenation of the patient 32 using drastic invasion techniques.

A system, including its components, and methods of use of the system, for quickly dislodging the foreign body 60, and facilitating removal thereof from the patient 32, is described and claimed below. The system provides a new modality to relieve an obstructed airway, and allows a caregiver, with minimal training, to proceed quickly, and even blindly without a light source, to minimize airway trauma, relieve airway obstruction, and provide oxygenation.

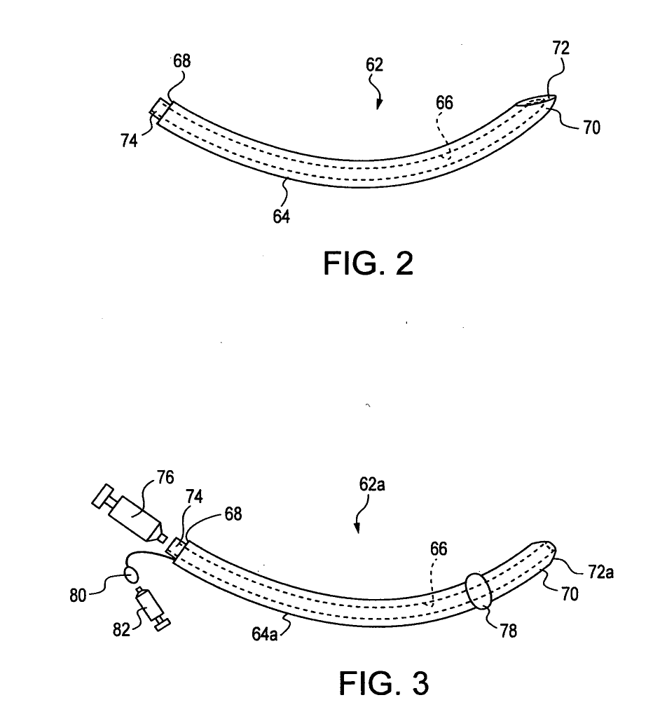

Referring to FIG. 2, a first embodiment of a retrograde device, such as, for example, a retrograde intubation device 62, is a component of the above-noted system, and includes a malleable retrograde member. A first embodiment of the retrograde member is a retrograde tube 64 having a hollow passage or opening 66 extending through the tube from a proximal end 68 to a distal end 70 thereof. In this embodiment, the retrograde tube 64 is formed with a uniform outer diameter from the proximal end 68 to the distal end 70.

In a second embodiment of the retrograde device, the retrograde member is solid, but is configured externally in the same manner as the external configuration of the retrograde tube 64.

The distal end 70 of the retrograde tube 64 is formed with a bevelled distal surface 72, to form a membrane piercing tip at the distal end. The proximal end 68 of the retrograde tube 64 is formed with an adapter 74, for receipt of external facilities. The malleability of the tube 64 facilitates reshaping of the tube, on sight, where necessary to accommodate potentially different structural arrangements of the oral cavity 36 and the pharyngeal area 38 of different patients 32. Generally, the tube 64 can be formed to the curved shape illustrated in FIG. 2, with minor variations in curvature.

Referring to FIG. 3, a second embodiment of a retrograde intubation device 62a is similar to the retrograde intubation device 62 shown in FIG. 2. For those features which are common in the devices 62 and 62a, the same numerals will be assigned to such common features. A retrograde tube 64a of the device 62a is formed with a uniform outer diameter in the same manner as the retrograde tube 64 of the device 62. The distal end 70 of the tube 64a is formed with a generally sharp surface 72a in a pencil-point configuration, to provide for an enhanced membrane piercing tip.

In addition, a syringe 76 is positioned for assembly with the adapter 74. A miniature inflatable cuff 78, in a deflated state, can be optionally attached to, and about, an intermediate portion of the outer surface of the tube 64a, near the distal end 70 thereof, as an optional component of the above-noted system. A pilot balloon 80 is assembled with the tube 64a, and a syringe 82 is assembled with the pilot balloon, to facilitate selective inflation of the cuff 78.

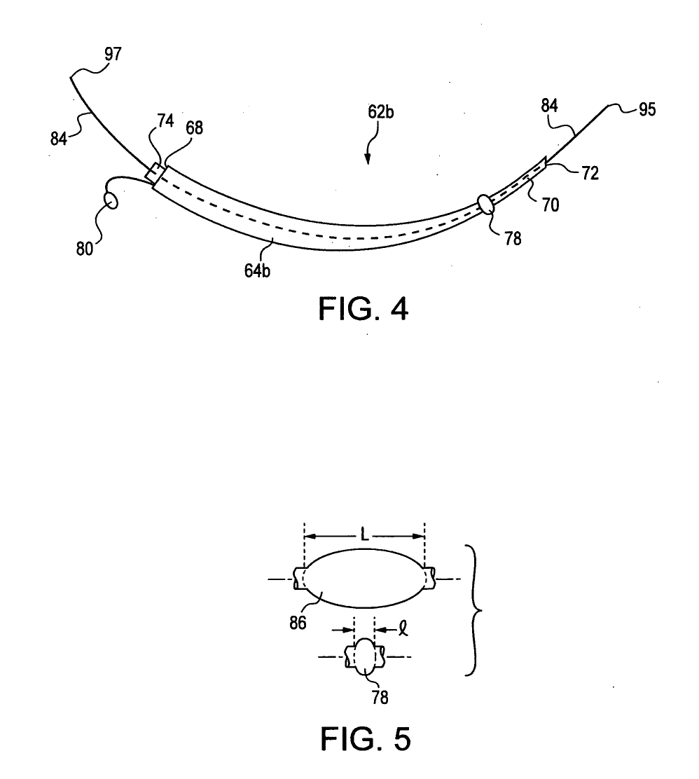

As shown in FIG. 4, a third embodiment of a retrograde intubation device 62b is similar to the retrograde intubation devices 62 and 62a, shown in FIGS. 2 and 3, respectively. For those features which are common in the devices 62, 62a and 62b, the same numerals will be assigned to such common features. A retrograde tube 64b of the device 62b is formed with a hollow opening in the same manner as the opening 66 of the tube 64 (FIG. 2), but is not shown. The body of the tube 64b is tapered from the proximal end 68, of a larger diameter, to the distal end 70, of a smaller diameter, and is formed with the bevelled surface 72. The miniature inflatable cuff 78 also can be optionally attached to, and about, the intermediate portion of the outer surface of the tapered tube 64b near the distal end 70, as an optional component of the above-noted system.

A guidewire 84, which is a component of the above-noted system, is located within the hollow opening 66 (FIGS. 2 and 3) of the tube 64b, with opposite ends extending from the proximal end 68 and the distal 70 of the tube. The guidewire 84 can also be inserted into the hollow openings 66 of the tubes 64 and 64a, when such tubes are used as a component of the above-noted system. While the guidewire 84 can be inserted initially into the opening 66 at either the proximal end 68 or the distal end 70 of the tubes 64, 64a and 64b, it is preferable that the guidewire be initially inserted into the proximal end.

Referring to FIG. 5, illustrated are the relative sizes of a conventional cuff 86, typically used with present day airway devices, and the miniature inflatable cuff 78, the size of which was heretofore believed to be unknown and unavailable, and which can be attached optionally on the retrograde tubes 64, 64a and 64b of the above-noted system, as described above. The typical conventional cuff 78 generally has an axial length “L” of approximately 3.0 cm. For reasons noted below, the axial length “l” of the miniature cuff 78 is within a range of 0.75 cm. to 1.25 cm., with a preferable axial length “l” being 1.0 cm.

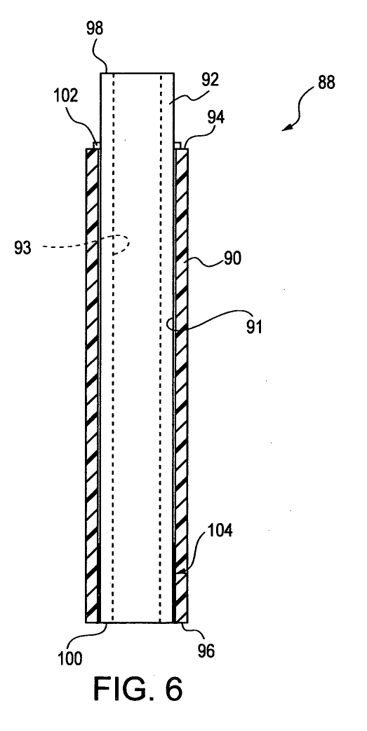

As shown in FIG. 6, a carrier device 88 is a component of the above-noted system, and, in a first embodiment thereof, the device is solid.

In a second embodiment of the carrier device 88, the device is formed by an outer cylindrical sleeve 90, and an inner cylindrical sleeve 92. The inner diameter of the outer sleeve 90 is slightly greater than the outer diameter of the inner sleeve 92 such that the sleeves are arranged for telescoping movement relative to each other.

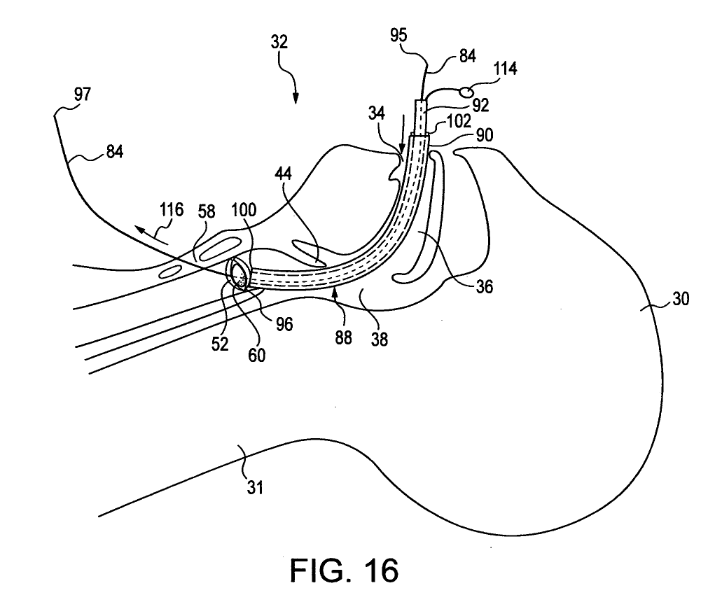

The outer sleeve 90 is formed with a proximal end 94 and a distal end 96, while the inner sleeve 92 is formed with a proximal end 98 and a distal end 100. The outer sleeve 90 and the inner sleeve 92 are formed of a malleable material which allows the caregiver to configure the outer and inner sleeves, and thereby the carrier device 88, to a desired shape, curved or straight. This feature allows the carrier device 88 to be formed in an straight axial alignment, or in a curved axial alignment, as shown in FIG. 16, without departing from the spirit and scope of the invention.

The outer sleeve 90 is formed with a hollow cylindrical passage or opening 91, which extends from the proximal end 94 to the distal end 96 thereof. The inner sleeve 92 is formed with a hollow cylindrical passage or opening 93, which extends from the proximal end 98 to the distal end 100 thereof. In this embodiment, the carrier device 88 is a hollow foreign-body expelling device.

A retaining clip 102 is situated at the proximal end 94 of the outer sleeve 90, and is in engagement with an intermediate portion of the inner sleeve 92 near the proximal end 98 thereof. The retaining clip 102 retains the outer sleeve 90 and the inner sleeve 92 together when the sleeves are not being urged into telescoping movement.

As shown in FIG. 6, the inner sleeve 92 is axially longer than the outer sleeve 90, with a proximal portion of the inner sleeve extending above the proximal end 94 of the outer sleeve. It is noted that the outer sleeve 90 and the inner sleeve 92 could be of the same axial length without departing from the spirit and scope of the invention.

A foreign-body dislodging means 104 is attached to the distal end 100 of the inner sleeve 92, and extends toward the proximal end 98 of the inner sleeve. When the outer sleeve 90 and the inner sleeve 92 are arranged as shown in FIG. 6, the means is captured between the adjacent inner surface of the outer sleeve 90 and the outer surface of the inner sleeve, to which the means is attached. The dislodging means 104 is a component of the above-noted system.

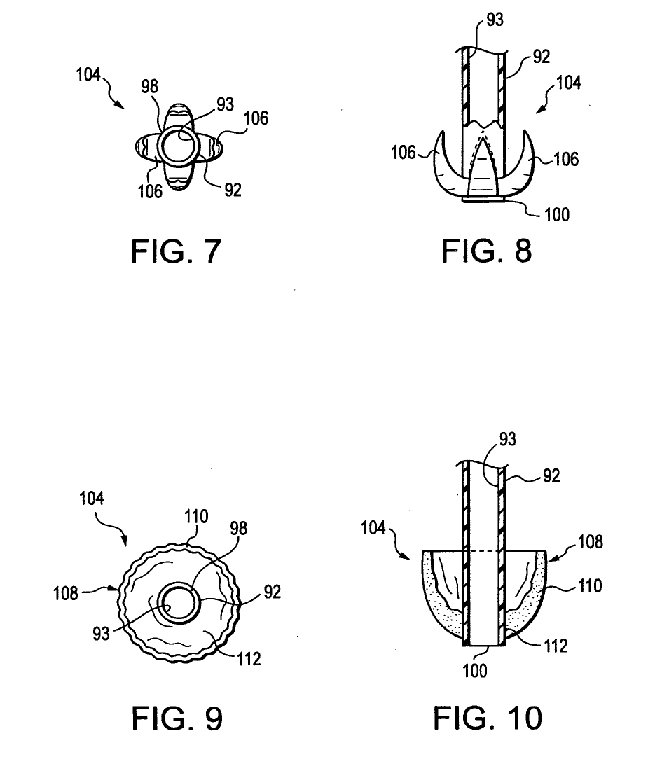

As shown in FIGS. 7 and 8, a first embodiment of the foreign-body dislodging means 104 is formed by a plurality of flexible fingers 106, which are attached to an outer surface of the inner sleeve 92, at the distal end 100 thereof, with the hollow cylindrical opening 93 remaining open at the distal end as well as at the proximal end 98. The flexible fingers 106 are normally biased to extend outward from the adjacent outer surface of the inner sleeve 92 in a basket-like arrangement, in the manner shown clearly in FIG. 8, but are captured and pressed against the adjacent outer surface of the inner sleeve, when the outer sleeve 90 and the inner sleeve are assembled as shown in FIG. 6.

As shown in FIGS. 9 and 10, a second embodiment of the foreign-body dislodging means 104 is formed by an inflatable bowl-shaped cuff 108, which is attached to the outer surface of the inner sleeve 92, at the distal end 100, with the hollow cylindrical opening 93 remaining open at the distal end, as well as at the proximal end 98. When the cuff 108 is inflated, a side wall 110 and a base 112 thereof extend outward from the adjacent outer surface of the inner sleeve 92 in a bowl-like arrangement, in the manner shown clearly in FIG. 10, but is captured and pressed against the adjacent outer surface of the inner sleeve, when the cuff is in the deflated mode, and the outer sleeve 90 and the inner sleeve are assembled as shown in FIG. 6.

As shown in FIG. 11, the foreign body 60 is lodged undesirably between the vocal cords 52, at the glottic opening 50 to the trachea 48, of the patient 32. In a first step of using the system to dislodge the foreign body 60, the bevelled distal end 70 of the retrograde tube 64 of the retrograde intubation device 62 is poised to penetrate, at a point of penetration, the cricoidthyroid membrane 58 of the patient. Any of the devices 62, 62a or 62b could be used for this procedure, without departing from the spirit and scope of the invention. Also, if desired, and prior to the penetration of the tube 64 into the cricoidthyroid membrane 58, a small nick could be made at the site of the subsequent penetration of the tube, with a regular needle or scalpel, to enhance the entry of the distal end 70 into the membrane, without departing from the spirit and scope of the invention.

It is noted that the cricoidthyroid membrane 58 is an area where conventional intubation may be initiated, where a large intubation tube (not shown) is directed through a significantly invasive, previously-formed incision through the membrane, and downward into the trachea 48 toward the lungs, thereby bypassing the glottic opening 50 and the vocal cords 52 where the foreign body 60 may be lodged. Thereafter, ventilation facilities must be attached to the large intubation tube, externally of the patient's neck 31, in the area of the incision, to maintain ventilation and oxygenation, while other means must still be used to dislodge and remove any foreign body 60 which may be lodged between the vocal cords 52.

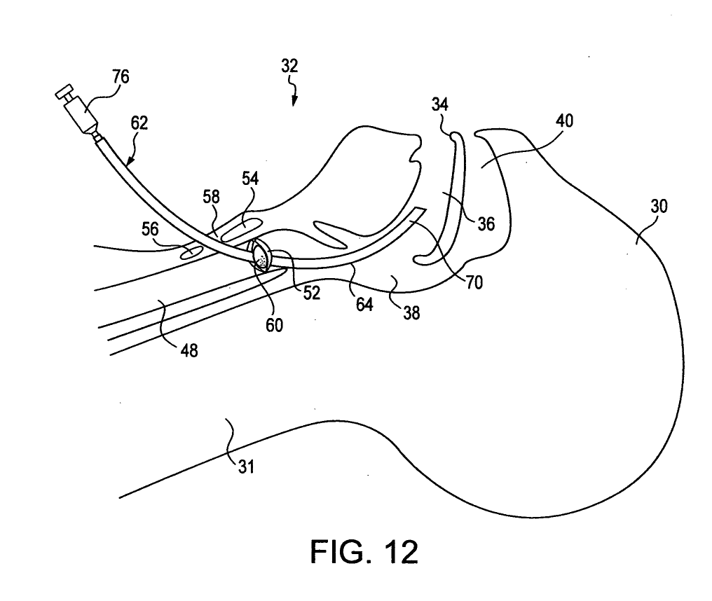

Referring to FIG. 12, the distal end 70 of the tube 64 has penetrated through the cricoidthyroid membrane 58, and has been moved into the trachea 48. When the distal end 70 of the tube 64 enters the trachea 48, a pressure change occurs at the distal end of the tube, which is sensed at the syringe 76 by movement of the stem of the syringe, and/or reaction of fluid within the barrel of the syringe. At this point, the caregiver knows that the distal end 70 of the tube 64 has penetrated into the trachea.

Due to the curvature of the tube 64, continued movement of the tube advances the distal end 70 of the tube upward between the vocal cords 52 and the foreign body 60, but not through the foreign body, and farther into the pharyngeal area 38 and the oral cavity 40. If the caregiver senses difficulty in advancing the distal end 70 of the tube 64 during this step, the tube may be removed and, due to the malleability of the tube, the caregiver may reshape the tube into a different curvature, and then the insertion procedure.

Also, during this step of the process, the foreign body 60 may be lodged so thoroughly between the vocal cords 52, that the distal end 70 of the retrograde tube 64 may not be able to push the lodged foreign body, whereby the distal end of the tube slides between the foreign body and the vocal cords, as shown in FIG. 12, and ultimately into the pharyngeal area 38 and the oral cavity 40.

However, it is possible that, if the foreign body 60 was not so thoroughly lodged, the distal end 70 of the moving tube 64 may dislodge and push the foreign body into the pharyngeal area to then allow ventilation and oxygenation of the patient 32, and oral removal of the dislodged foreign body.

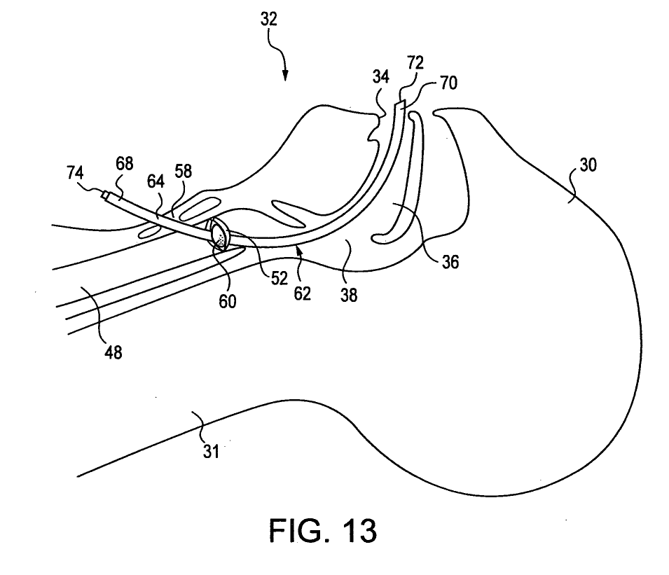

As shown in FIG. 13, the tube 64 has been moved farther through the cricoidthyroid membrane 58, such that the distal end 70 is extending out of the oral cavity opening 34, while the proximal end 68 of the retrograde tube 64 remains outside of the cricoidthyroid membrane. An intermediate portion of the tube 64 now resides between the vocal cords 52 and the lodged foreign body 60.

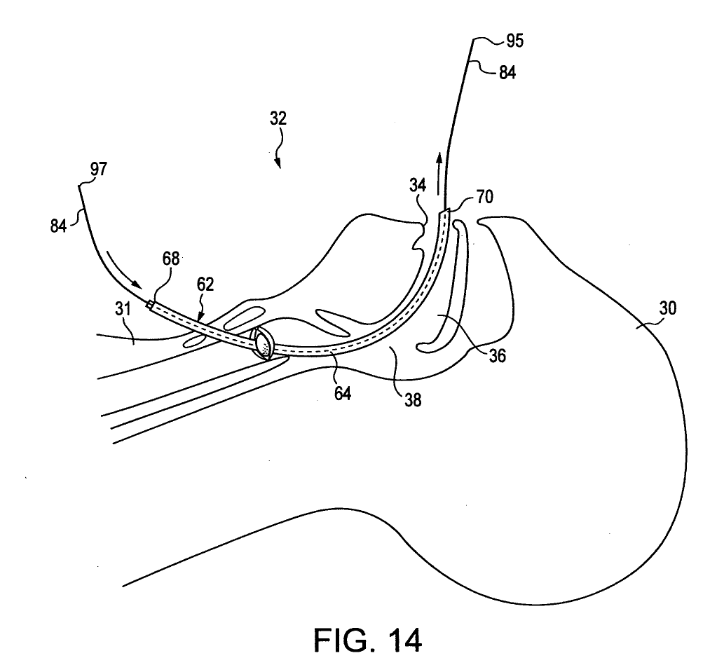

Referring to FIG. 14, the guidewire 84 is inserted into the hollow opening 66 (FIG. 2) at the proximal end 68 of the tube 64. The guidewire 84 is then moved through the hollow opening 66 of the retrograde tube 64, with a distal or leading end 95 of the guidewire extending out of the hollow opening at the distal end 70 of the retrograde tube, and with a proximal or trailing end 97 of the guidewire extending out of the hollow opening at the proximal end 68 of the retrograde tube. While the guidewire 84 can be inserted into the hollow opening 66 of the tube 64 from either the proximal end 68 or the distal end 70, it is preferable that the guidewire be initially inserted into the hollow opening from the proximal end.

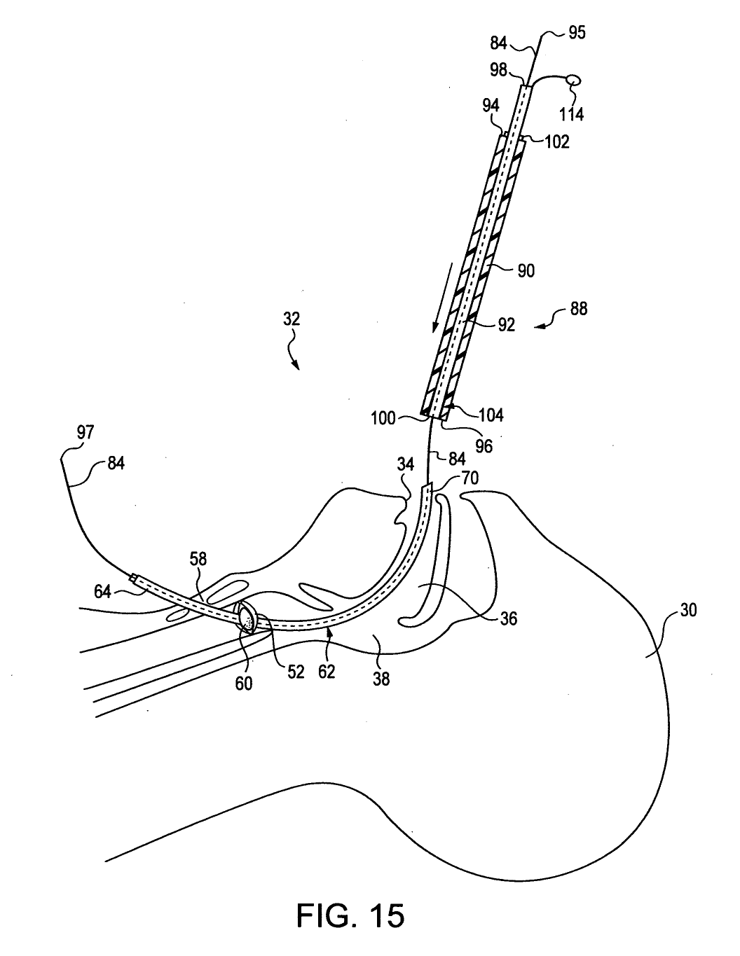

As shown in FIG. 15, the hollow cylindrical opening 93, at the distal end 100 of the inner sleeve 92 of the carrier device 88, is initially positioned over the leading end 95 of the guidewire 84. The carrier device 88 is then moved over the guidewire 84, toward the oral cavity opening 34 of the patient 32, with the distal end 100 initially approaching the oral cavity opening, and the proximal end 98 of the inner sleeve trailing behind. In FIG. 15, either the above-noted first embodiment of the foreign-body dislodging means 104, which includes the flexible fingers 106 (FIGS. 7 and 8), or the second embodiment, which includes the inflatable bowl-shaped cuff 108 (FIGS. 9 and 10) and a pilot balloon 114, can be used.

Referring to FIG. 16, the carrier device 88 has been moved through the oral cavity opening 34, with the hollow cylindrical opening 93 of the inner sleeve 92 being guided over the retrograde intubation device 62, and the carrier device 88 conforming to the curvature of the device 62. As shown in FIG. 16, the carrier device 88 has been moved over the retrograde tube 64 and the guidewire 84 to a position whereat the distal ends 96 and 100 of the outer sleeve 90 and the inner sleeve 92, respectively, are located adjacent a portion of the lodged foreign body 60, which faces the epiglottis 44, i.e., the epiglottis side of the lodged foreign body.

It is noted that, during this maneuver, the distal ends 96 and 100 of the outer sleeve 90 and the inner sleeve 92, respectively, have been directed and guided to the location where the tube 64 of the retrograde intubation device 62, and the guidewire 84, were previously moved between the foreign body 60 and the vocal cords 52, as described above, and as shown, for example, in FIG. 14.

Thereafter the clip 102 is removed to allow relative movement between the outer sleeve 90 and the inner sleeve 92. Upon subsequent additional slight movement of the inner sleeve 92 toward the trachea 48, the distal end 100 of the inner sleeve is guided over the retrograde tube 64, and slightly into the trachea, so that the distal end 100 is now past the side of the foreign body 60 which faces the lungs, i.e., the lung side of the foreign body, as shown in.

After the carrier device 88 has been located as described above, the retrograde intubation device 62 is withdrawn, over the guidewire 84, through the membrane 58, and completely from the head 30 and the neck 31 of the patient 32. This is accomplished by the caregiver grasping the proximal end 68 of the retrograde tube 64 and moving the device 62 outward through the cricoidthyroid membrane 58 in the direction of arrow 116. The distal end 100 of the inner sleeve 92 remains over the guidewire 84, and within the trachea 48, on the lung side of the foreign body 60.

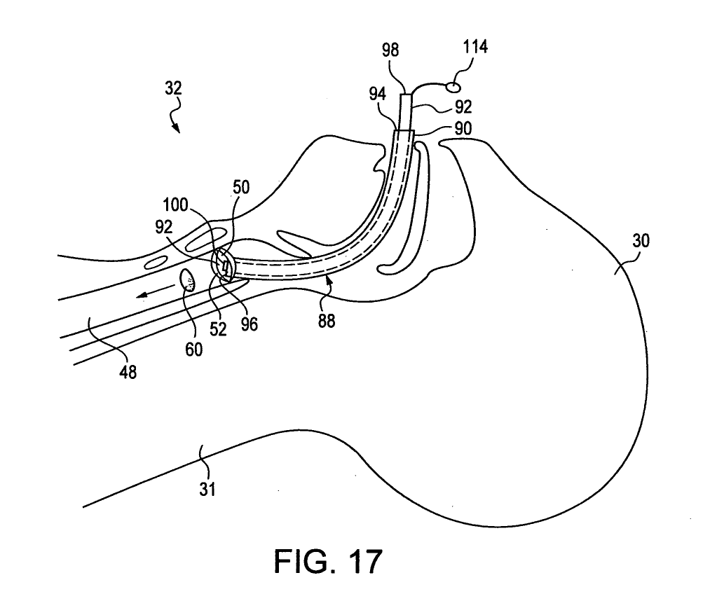

Referring to FIG. 17, thereafter the guidewire 84 is withdrawn in the same manner that the retrograde intubation device 62 was withdrawn, that is, by the caregiver grasping the trailing end 97 (FIG. 16) of the guidewire and moving the guidewire in the direction of arrow 116 (FIG. 16). In this manner, the guidewire 84 is completely withdrawn from the hollow cylindrical opening 93 of the inner sleeve 92, at the distal end 100 thereof, through the cricoidthyroid membrane 58, and completely from the head 30 and the neck 31 of the patient 32. It is noted that the guidewire 84 can also be withdrawn from the hollow cylindrical opening 93 of the inner sleeve 92, at the proximal end 98 thereof, without departing from the spirit and scope of the invention.

During removal of the guidewire 84, the carrier device 88 remains in the position illustrated in FIG. 16. Thereafter, the inner sleeve 92 is moved farther into the trachea 48, relative to the outer sleeve 90, by pushing the proximal end 98 of the inner sleeve farther toward the proximal end 94 of the outer sleeve.

As the inner sleeve 92 is moved within the outer sleeve 90, the distal end 100 of the inner sleeve is moved out of the hollow cylindrical opening 91 at the distal end 96 of the outer sleeve, and between the lodged foreign body 60 and the vocal cords 52. As a result of this movement of the inner sleeve 92, it is possible that the foreign body 60 may become dislodged and be moved farther into the trachea 48. In this instance, upon continued movement of the inner sleeve 92 toward the trachea 48, the distal end 100 of the inner sleeve is moved farther into the trachea to move the foreign body 60 toward, and into, the lungs, thereby removing the obstruction, and allowing ventilation and oxygenation of the patient 32. The foreign body 60 can then be removed from the lungs by conventional surgical procedures in a hospital environment.

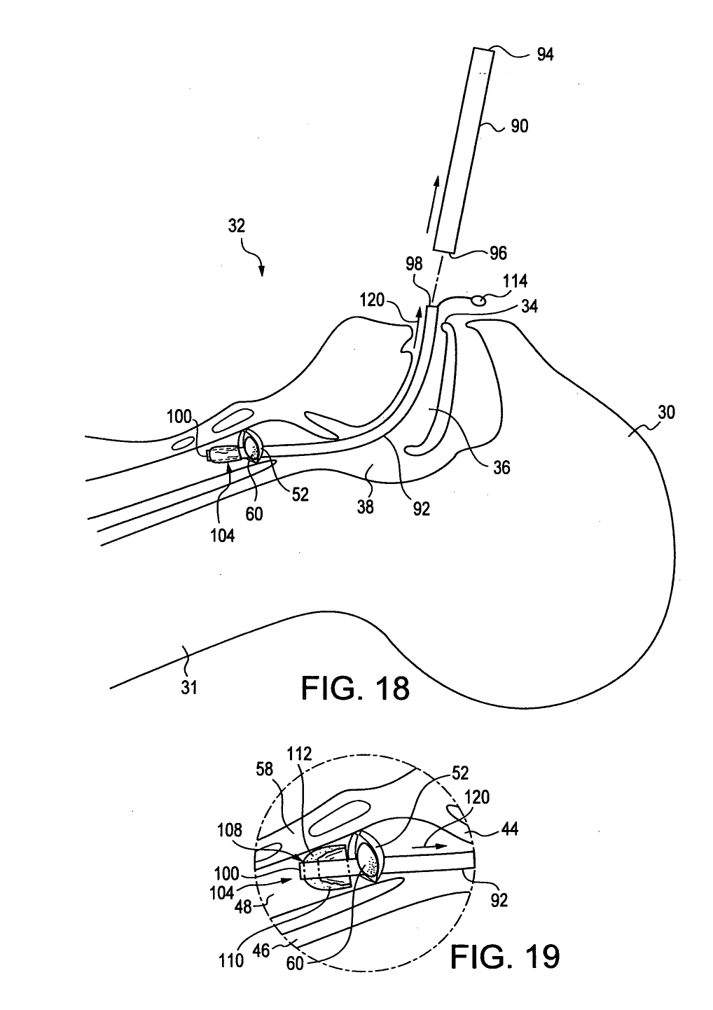

Referring to FIG. 18, if the foreign body 60 does not become dislodged farther into the trachea 48 in the manner noted above, but remains lodged within the vocal cords 52, continued movement of the inner sleeve 92 toward the trachea 48 will place the distal end 100 of the inner sleeve farther into the trachea, and past the lodged foreign body. Eventually, the distal end 100 of the inner sleeve 92, and the foreign-body dislodging means 104, will be located farther into the trachea 48 and fully past the lung side of the foreign body, as shown in FIG. 18. The outer sleeve 90 is now removed from the inner sleeve 92, and out of the oral cavity opening 34, as shown in FIG. 18.

If the first embodiment of the dislodging means 104, i.e., the flexible fingers 106, is attached to the distal end 100 of the inner sleeve 92, when the flexible fingers are moved out of the outer sleeve 90, between, and past, the vocal cords 52 and the foreign body 60, the biased fingers spring outward from the inner sleeve to the position shown in FIG. 8 to provide the basket-like arrangement.

Thereafter, the inner sleeve 92 is moved, or withdrawn, in the direction of arrow 120, and outward from the oral cavity opening 34. As the inner sleeve 92 is being withdrawn in this manner, the basket-like arrangement of the outwardly biased flexible fingers 106 is moved into engagement with the lung side of the lodged foreign body 60. Upon continued withdrawal of the inner sleeve 92, the flexible fingers 106 dislodge and move the foreign body 60 toward the pharyngeal area 38, and subsequently out of oral cavity 36 and the oral cavity opening 34. As the foreign body 60 is dislodged from the area of the vocal cords 52, the airway of the patient 32 is cleared sufficiently to ventilate and oxygenate the patient.

It is noted that, in the event that the dislodged foreign body 60 becomes detached from the basket-like arrangement of the flexible fingers 106 during the period when the foreign body is being moved through the pharyngeal area 38 and the oral cavity 36, the foreign body can be extracted by the caregiver. This extraction can be accomplished by the caregiver grasping the foreign body 60, by use of the caregiver's fingers or a conventional grasping instrument, and withdrawing the foreign body through the oral cavity opening 34, without departing from the spirit and scope of the invention.

Referring to FIG. 19, the second embodiment of the foreign-body dislodging means 104, i.e., the inflatable bowl-shaped cuff 108, is used, instead of the flexible fingers 106. After the deflated cuff 108 has been moved into the trachea 48, and past the lung side of the foreign body 60, as shown in FIGS. 18 and 19, the pilot balloon 114 is used to inflate the cuff to the condition shown in FIGS. 10 and 19.

Thereafter, the inner sleeve 92 is moved, or withdrawn, in the direction of the arrow 120, and outward from the oral cavity opening 34. As the inner sleeve 92 is being withdrawn in this manner, the open portion of the bowl-shaped arrangement of the inflated cuff 108 captures the lung side of the lodged foreign body 60, and dislodges and withdraws the foreign body toward the pharyngeal area 38, and subsequently out of oral cavity 36 and the oral cavity opening 34.

As the foreign body 60 is dislodged from the area of the vocal cords 52 by the inflated cuff 108, the airway of the patient 32 is cleared sufficiently to ventilate and oxygenate the patient. It is noted that, in the event that the dislodged foreign body 60 becomes detached from the bowl-shaped arrangement of the inflated cuff 108 during the period when the foreign body is being moved through the pharyngeal area 38 and the oral cavity 36, the foreign body can be extracted by the caregiver. This extraction can be accomplished, in the manner described above, by the caregiver grasping the foreign body 60, by use of the caregiver's fingers or a conventional grasping instrument, and thereafter withdrawing the foreign body through the oral cavity opening 34, without departing from the spirit and scope of the invention.

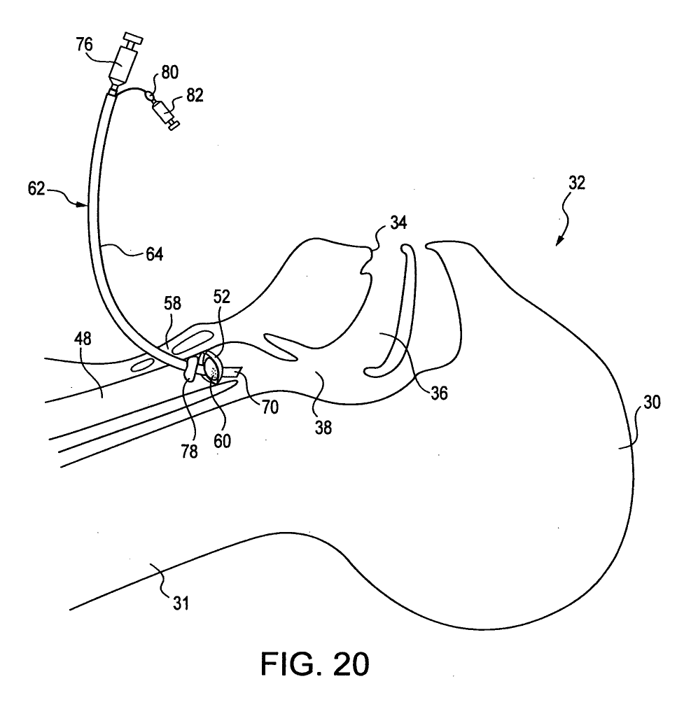

Referring to FIG. 20, in another technique for dislodging the foreign body 60 from the area of the vocal cords 52, by use of the above-noted system, the retrograde intubation device 62, with the miniature inflatable cuff 78 (FIGS. 4 and 5), in the deflated state, is inserted through the cricoidthyroid membrane 58 in the same manner described above with respect to the insertion of the device 62 without the cuff 78, and with reference to FIGS. 11 and 12.

During the period when the portion of the retrograde tube 64, to which the miniature deflated cuff 78 is attached, is being moved through the cricoidthyroid membrane 58, the caregiver will experience, and overcome, a low-level opposition to the insertion of the tube, and continue with the insertion procedure.

When the miniature deflated cuff 78 enters the trachea 48, the caregiver experiences a relief from the low-level opposition, whereafter the tube 64 more freely moves through the membrane 58. Upon sensing the relief from the opposition to the insertion of the tube 64 into the membrane 58, the caregiver recognizes that the deflated cuff 78 has cleared the membrane 84, and is now in the trachea 48. Also during this period, the distal end 72 of the tube 64 has been moved into the trachea 48 and between the lodged foreign body 60 and the vocal cords 52, in the manner described above.

After the deflated miniature cuff 78 has been moved into the trachea 48, as described above, the pilot balloon 80 is activated, by use of the syringe 82, to inflate the miniature cuff, as shown in FIG. 20.

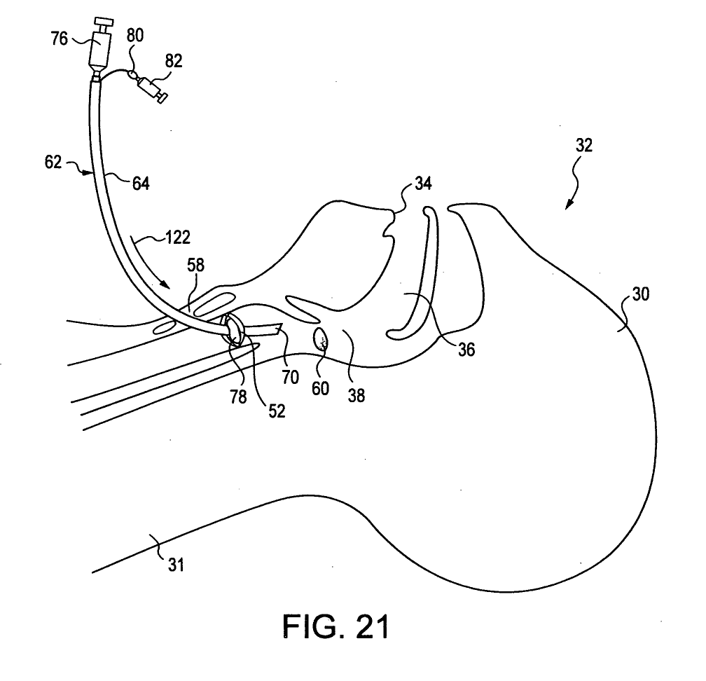

Referring to FIG. 21, after the inflation of the miniature cuff 78, the tube 64 is moved farther in the distal direction, as shown by arrow 122, whereby the inflated miniature cuff 78 engages the lung side of the foreign body 60, and urges the foreign body from between the vocal cords 52, and into the pharyngeal area 38. At this time, the airway of the patient 32 is cleared and the patient can be ventilated and oxygenated. The foreign body 60 can now be extracted by the caregiver grasping, or using a grasping instrument to grasp, the dislodged foreign body, and withdrawing the foreign body through the oral cavity opening 34, as described above, without departing from the spirit and scope of the invention.

The miniature cuff 78 is then deflated, and the tube 64 of the retrograde intubation device 62, with the deflated cuff 78, is withdrawn through the cricoidthyroid membrane 58 to complete the procedure of dislodging, and removing, the foreign body 60 from the patient 32.

The cricoidthyroid membrane 58 is relatively thin, which establishes the limited distance through which the deflated miniature cuff 78 has to travel to move from a location immediately outside the neck 31, at the point of initial penetration of the membrane 58 by the distal end 70 of the tube 64, to a location within the trachea 48, as described above. If the conventional cuff 86 (FIG. 5), at a length of 3.0 cm., was used instead of the miniature cuff 78, during the process of inserting the tube 64 through the cricoidthyroid membrane 58, leading portions of the length of the cuff 86 would eventually be located in the trachea 48, while trailing portions of the length of the cuff 86 would still be located within the passage formed in the membrane by inserting the tube 64.

If an attempt was made to inflate the conventional cuff 86 at this time, the portions of the passage in the membrane 58, in which the trailing portions of the cuff are located, could be affected to resulting in serious trauma to the patient 32. Therefore, by using the previously-unavailable miniature cuff 78, the cuff is capable of locating completely within the trachea 48 before the cuff is inflated.

In addition, it is preferable that any cuff 78 that is used in the above described process, with reference to FIGS. 20 and 21, be inflatable, when outside of the patient 32, to a transaxial size which is generally no greater than the diameter of the patient's trachea 48. However, the cuff 78 could be normally inflatable, i.e., when outside of the patient 32, to a transaxial size which is greater than the diameter of the trachea 48, without departing from the spirit and scope of the invention.

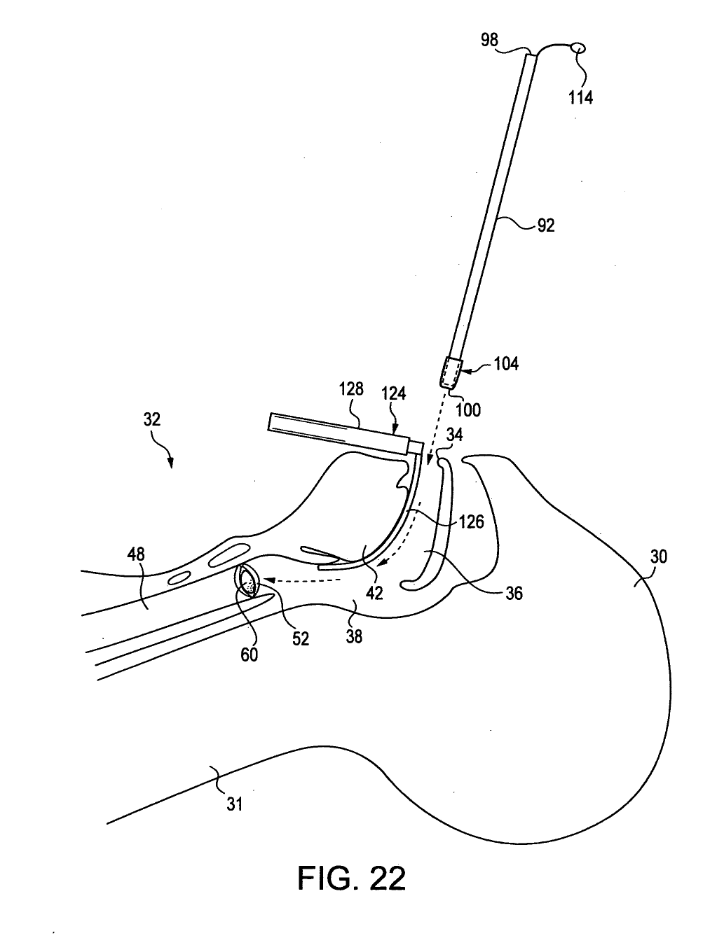

Referring to FIG. 22, a laryngoscope 124, including a blade 126 and a handle 128, can be inserted conventionally into the oral cavity 36 and the pharyngeal area 38 to provide direct visual observation, for a visually-observing caregiver, of the vocal cords 52 and the epiglottis side of the foreign body 60. The carrier device 88 (FIG. 6), or the inner sleeve 92 thereof in a stand-alone arrangement independent of the outer sleeve and with the foreign-body dislodging means 104, as shown in FIG. 22, or a solid rod element with the foreign-body dislodging means, can then be inserted directly into the oral cavity 36 and the pharyngeal area 38, by the visually-observing caregiver, to position the distal end of the device 88, or the distal end 100 of the stand-alone inner sleeve 92, adjacent the vocal cords 52 and the epiglottis side of the foreign body 60. The visually-observing caregiver then moves the inner sleeve 92, relative to the outer sleeve 90 if the inner sleeve is in assembly with the outer sleeve, or the distal end 100 of the stand-alone inner sleeve 92, into the trachea 48, between the vocal cords 52 and the foreign body 60 in the manner described above. Removal of the foreign body 60 then continues, in the manner as described above, by use of the foreign-body dislodging means 104.

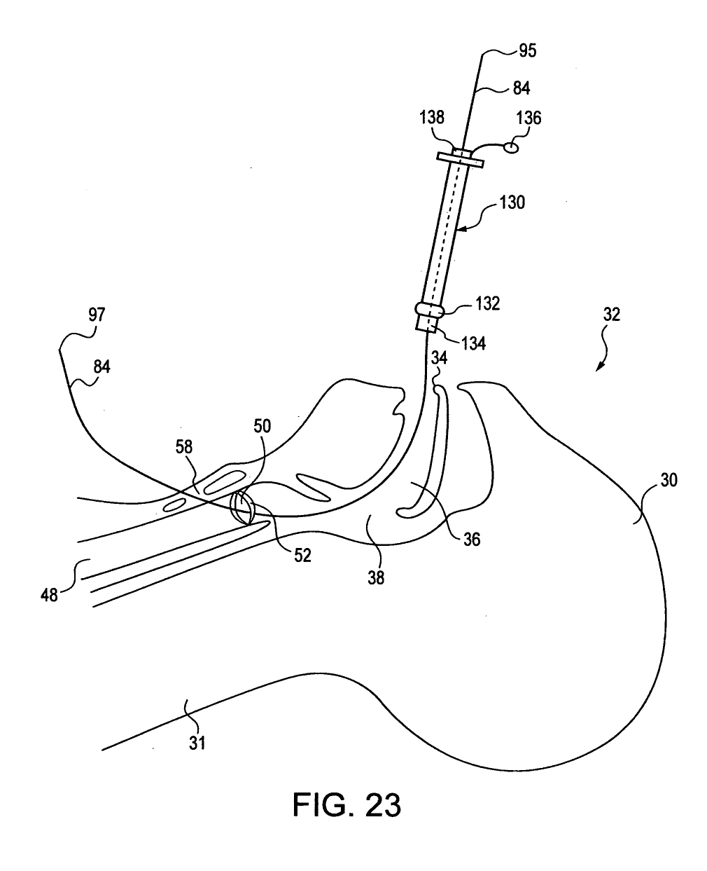

Referring to FIG. 23, the above-noted system can be used to intubate the patient 32, whose airway is not obstructed by the foreign body 60, by using the retrograde intubation device 62, in the manner described above and shown in FIG. 15, to facilitate placement of the guidewire 84 as shown. Thereafter, a hollow device, such as a conventional endotracheal tube 130, having a deflated inflatable cuff 132 at a distal end 134, and a pilot balloon 136 at a proximal end 138, can be positioned over the proximal end 95 of, and onto, the guidewire 84, and moved over the guidewire through the oral cavity 36 and the pharyngeal area 38 to a location where ventilation can be provided for the patient 32 in the conventional manner. It is noted that the endotracheal tube 130 may be formed in a conventional curved configuration, and/or be malleable to facilitate on site forming in a curved configuration, without departing from the spirit and scope of the invention.

Referring again to FIGS. 13 and 14, in another technique for using the system for providing an open airway, the retrograde intubation device 62 is mounted as described above, with the proximal end 70 extending out of the patient's oral cavity opening 34, and with the distal end 68 extending out of the point of penetration of the membrane 58. Thereafter, the guidewire 84 is extended through the retrograde tube 64 to the position illustrated in FIG. 14.

With the guidewire 84 now in the position shown, the retrograde intubation device 62 is withdrawn, preferably through the membrane 58 in the manner described above. Now, only the guidewire 84 remains, extending from outside of the patient's neck 31, through the membrane 58, between the vocal cords 52 and the foreign body 60, through the pharyngeal area 38, through the oral cavity 36, and out of the oral cavity opening 34.

It is noted that, with this process, the retrograde intubation device 62 could also be removed from the patient 32 by extracting the device through the oral cavity opening 34, without departing from the spirit and scope of the invention.

Thereafter, the carrier device 88 is positioned over the proximal end 95 of the guidewire 84, and moved over the guidewire toward the oral cavity opening 34 to the position illustrated in FIG. 15. With the retrograde intubation device 62 having been previously removed, as described above, the carrier device 88 is moved farther over the guidewire 84 and into the oral cavity 36 and the pharyngeal area 38 to the position shown in FIG. 16. The clip 102 is then removed, and the inner sleeve 92 of the carrier device 88 is then moved relative to the outer sleeve 90 to position the distal end 70 of the inner sleeve into the trachea 48 as described above, and as shown in FIG. 18. The outer sleeve 90 of the carrier device 88 is then withdrawn as described above, and as shown in FIG. 18. Thereafter, the dislodgement and removal of the foreign body 60 is accomplished as described above, by use of the foreign-body dislodging means 104.

Referring again to FIG. 13, in another technique for using the system for providing an open airway, the retrograde intubation device 62, or a solid rod element having an external configuration similar to the device 62, is mounted as described above, with the proximal end 70 extending out of the patient's oral cavity opening 34, and with the distal end 68 extending out of the point of penetration of the membrane 58. In this process, the guidewire 84 is not used.

Thereafter, the carrier device 88 is positioned outside of the oral cavity opening 34, similar to the position of the device as shown in FIG. 15, but without the presence of the guidewire 84. The distal end of the carrier device 88 is moved toward the distal end 70 of the retrograde tube 64, or the solid rod element, whereafter the hollow cylindrical opening 93 of the inner sleeve 92 is moved over the retrograde tube, or the solid rod element, to the position where the distal end of the dislodging device is located at the epiglottis side of the foreign body 60, similar to the position shown in FIG. 16, but without the presence of the guidewire 84.

The retrograde intubation device 62, or solid rod element, is then removed from the patient 32, in the manner described above, and the clip 102 is removed, whereafter the inner sleeve 92 is moved relative to the outer sleeve 90 to locate the distal end 100, and the foreign-body dislodging means 104, in the trachea 48 as shown in FIG. 18. The outer sleeve 90 can then be removed as described above, and the inner sleeve withdrawn to dislodge and remove the foreign body 60.

In still other techniques, the inner sleeve 92 is removed from assembly with the outer sleeve 90, whereby the inner sleeve will be used independently of the outer sleeve, similar to the arrangement and use of the inner sleeve, as illustrated in FIG. 22.

In these other techniques, the retrograde intubation device 62 is located in the patient 32 as shown in FIG. 13, whereafter the guidewire 84 may, or may not, be assembled within the hollow opening 66 of the retrograde tube 64, as described below. Thereafter, the retrograde tube 64 may, or may not be, withdrawn, also as described below.

In a first of such other techniques, if the guidewire 84 is assembled with the retrograde tube 64, as shown in FIG. 14, the inner sleeve 92 is moved so that the hollow cylindrical opening 93, at the distal end 100, is moved over the proximal end 95 of the guide wire 84, and is guided and moved farther over the guidewire and the retrograde tube 64, to position the distal end 100 of the inner sleeve between the vocal cords 52 and the foreign body 60, and slightly within the trachea 48. Thereafter, the retrograde tube 64 is withdrawn, in the manner described above, and then the guidewire 84 is also withdrawn, and the dislodging and withdrawing of the foreign body 60 proceeds as described above.

In a second of such other techniques, if the retrograde tube 64 has been withdrawn prior to insertion of the inner sleeve 92 over the guidewire 84, then the inner sleeve is guided over the guidewire only, thereby facilitating the locating of the distal end 100 of the inner sleeve between the vocal cords 52 and the foreign body 60, and slightly into the trachea 48. Thereafter, the guidewire 84 is withdrawn from the patient 32, and the dislodging and withdrawal of the foreign body 60 proceeds as described above.

In a third of such other techniques, if the retrograde tube 64 is inserted into the patient 32 as described above, and the guidewire 84 is not used, the inner sleeve 92 is inserted over, and guided by, the retrograde tube 64 only, thereby facilitating the locating of the distal end 100 of the inner sleeve between the vocal cords 52 and the foreign body 60, and slightly into the trachea 48. Thereafter, the retrograde tube 64 is withdrawn from the patient 32, and the dislodging and withdrawal of the foreign body 60 proceeds as described above.

The above-described first, second and third of such other techniques, in which the retrograde tube 64 may, or may not, be used, and the guidewire 84 may, or may not, be used, can also be applied to the insertion and guidance of the endotracheal tube 130 (FIG. 23).

As described above, system for providing an open airway, and the methods of use thereof, readily facilitates the a clearing of a patient's airway where the patient is lacking ventilation and oxygenation, and further enhances the insertion of an endotracheal tube into the patient's airway. In addition, the system and methods of use thereof allows a caregiver, with limited training, the ability to ventilate and oxygenate the patient in a non-hospital environment. This is significantly important where the patient's airway has been obstructed, particularly in a non-hospital environment, and prompt clearance of the airway is necessary to sustain the life of the patient.

In general, the above-identified embodiments are not to be construed as limiting the breadth of the present invention. Modifications, and other alternative constructions, will be apparent which are within the spirit and scope of the invention as defined in the appended claims.

What is claimed is:

1. In a system for providing an open airway for a patient, where the patient has a neck, a cricoidthyroid membrane, a glottic opening at the vocal cords, a pharyngeal area, an oral cavity and an oral cavity opening, which comprises:
a retrograde device positionable through the cricoidthyroid membrane, the glottic opening at the vocal cords, the pharyngeal area, the oral cavity and at least to a location outside of the patient's oral cavity opening; and
a carrier device having an end guidable along the retrograde intubation device from the location outside of the patient's oral cavity opening, through the oral cavity opening, the oral cavity, the pharyngeal area, and at least into a position adjacent the glottic opening.

2. In the system as set forth in claim 1, where the retrograde intubation device comprises:
a retrograde member formed with a membrane piercing tip at an end thereof.

3. In the system as set forth in claim 2, wherein the end of the retrograde member is a first end, and the retrograde member further comprises:
a hollow passage extending from, and through, the first end to, and through, a second end of the retrograde member spaced from the first end.

4. In the system as set forth in claim 2, wherein the retrograde member is formed with a uniform external diameter from the first end to the second end of the retrograde member.

5. In the system as set forth in claim 2, wherein the retrograde member is tapered from the first end having a prescribed diameter to the second end having a diameter larger than the prescribed diameter.

6. In the system as set forth in claim 2, wherein the membrane piercing tip is formed by a bevelled surface formed across the first end of the retrograde member.

7. In the system as set forth in claim 2, wherein the membrane piercing tip is formed by a generally sharp pencil-point configuration at the first end of the retrograde member.

8. In a system as set forth in claim 2, wherein the retrograde member is formed in a curved configuration.

9. In a system as set forth in claim 2, wherein the retrograde member is formed of a malleable material to facilitate forming the retrograde member in a selected curvature.

10. In a system as set forth in claim 2, wherein the end of the retrograde member is a first end, which further comprises:
a miniature inflatable cuff is attached about an intermediate outer portion of the retrograde member, with the cuff being closer to the first end than to the second end of the retrograde member.

11. In the system as set forth in claim 10, wherein the miniature cuff is formed with an axial length which is within a range of 0.75 cm. to 1.25 cm.

12. In the system as set forth in claim 10, wherein the miniature cuff is formed with an axial length of 1.0 cm.

13. In the system as set forth in claim 10, wherein the miniature cuff is inflatable when outside of the patient to a transaxial size which is no greater than the diameter of a trachea of the patient.

14. In the system as set forth in claim 1, which further comprises:
a foreign-body dislodging means attached to the carrier device.

15. In a system as set forth in claim 14, wherein the foreign-body dislodging means comprises:
a plurality of spring-biased flexible fingers attached to and extendable from the carrier device.

16. In a system as set forth in claim 14, wherein the foreign-body dislodging means comprises:
an inflatable cuff attached to and extendable from the carrier device.

17. In the system as set forth in claim 1, wherein the carrier device comprises:
an outer sleeve formed with a hollow passage extending therethrough and having a prescribed transaxial configuration and a prescribed uniform transaxial dimension;
an inner sleeve formed with a hollow passage extending therethrough and with the prescribed transaxial configuration, and a uniform transaxial dimension which is less than the prescribed uniform transaxial dimension, and
the inner sleeve being located in, and movable within, the hollow passage of the outer sleeve.

18. In the system as set forth in claim 17, which further comprises:
a foreign-body dislodging means attached to an outer surface of the inner sleeve adjacent one end thereof.

19. In a system as set forth in claim 18, wherein the foreign-body dislodging means comprises:
a plurality of spring-biased flexible fingers attached to and extendable from the outer surface of the inner sleeve.

20. In a system as set forth in claim 18, wherein the foreign-body dislodging means comprises:
an inflatable cuff attached to and extendable from the outer surface of the inner sleeve.

21. In a system as set forth in claim 20, wherein the inflatable cuff, when inflated, forms a bowl-shaped configuration which opens away from the adjacent one end of the inner sleeve.

22. In the system as set forth in claim 1, wherein the carrier device is selected from the group consisting of a hollow foreign-body expelling device, a hollow inner sleeve, a hollow endotracheal tube, and a solid rod element.

23. In a system as set forth in claim 1, which further comprises:
the retrograde device formed with a hollow unobstructed passage extending from, and through, a first end to a second end of the retrograde device, and
a guidewire extendable through the unobstructed passage of the retrograde intubation device at least to the location outside of the patient's oral cavity opening.

24. In a system as set forth in claim 1, wherein the carrier device is formed of a malleable material to facilitate forming the carrier device in a selected curvature.

25. In a system for dislodging a foreign body to provide an open airway for a patient, where the patient has an oral cavity opening, an oral cavity, a pharyngeal area, a glottic opening, and the vocal cords, which comprises:
a rod element formed with a forward end, and
a foreign-body dislodging means attached to the forward end of the rod element.

26. In a system as set forth in claim 25, wherein the foreign-body dislodging means comprises:
a plurality of spring-biased flexible fingers attached to and extendable from the rod element.

27. In a system as set forth in claim 25, wherein the foreign-body dislodging means comprises:
an inflatable cuff attached to and extendable from the rod element.

28. In a system for providing an open airway for a patient, where the patient has a neck, a cricoidthyroid membrane, a glottic opening at the vocal cords, a pharyngeal area, an oral cavity and an oral cavity opening, which comprises:
a guidewire extendable through the cricoidthyroid membrane, the glottic opening at the vocal cords, the pharyngeal area, the oral cavity and at least to a location outside the patient's oral cavity opening; and
a carrier device having an end guidingly movable along the guidewire from the location outside of the patient's oral cavity opening, through the oral cavity opening, into the oral cavity and the pharyngeal area, and at least into a position adjacent the glottic opening.

29. A method of providing an open airway for a patient, where the patient has a neck, a cricoidthyroid membrane, a trachea, vocal cords, a glottic opening at the vocal cords, a pharyngeal area, an oral cavity and an oral cavity opening, which comprises the steps of:
positioning a retrograde device within, and through, the cricoidthyroid membrane, the glottic opening at the vocal cords, the pharyngeal area, the oral cavity and at least to a location outside of the patient's oral cavity opening; and
moving a carrier device guidingly along the retrograde device from the location outside of the patient's oral cavity opening, through the oral cavity opening, the oral cavity, the pharyngeal area, and at least into a position adjacent the glottic opening.

30. The method as set forth in claim 29, wherein the retrograde device is a retrograde intubation device.

31. The method as set forth in claim 29, wherein the retrograde device is a solid rod element.

32. The method as set forth in claim 29, wherein the carrier device is a hollow device.

33. The method as set forth in claim 29, which further comprises the step of:
withdrawing the retrograde device from within the patient.

34. The method as set forth in claim 29, which, prior to the step of moving the carrier device, further comprises the step of:
attaching a foreign-body dislodging means to the carrier device.

35. The method as set forth in claim 34, which further comprises the step of:
moving the carrier device to locate the foreign-body dislodging means through the pharyngeal area, the glottic opening at the vocal cords, and into the trachea.

36. The method as set forth in claim 34, which further comprises the step of:
moving the carrier device to move the foreign-body dislodging means through the pharyngeal area, between the vocal cords and any foreign body lodged within the vocal cords, and into the trachea.

37. The method as set forth in claim 35, which further comprises the step of:
moving the carrier device to withdraw the foreign-body dislodging means from within the trachea, and through the glottic opening at the vocal cords, to thereby dislodge any foreign body which may be located in the area of the vocal cords.

38. The method as set forth in claim 36, which further comprises the step of;
extracting the dislodged foreign body from within the patient.

39. A method of providing an open airway for a patient, where the patient has a neck, a cricoidthyroid membrane, vocal cords, a glottic opening at the vocal cords, a pharyngeal area, an oral cavity and an oral cavity opening, which comprises:
positioning a guidewire within, and through, the cricoidthyroid membrane, the glottic opening at the vocal cords, the pharyngeal area, the oral cavity and at least to a location outside of the patient's oral cavity opening; and
moving a carrier device guidingly along the guidewire from the location outside of the patient's oral cavity opening, through the oral cavity opening, the oral cavity, the pharyngeal area, and at least into a position adjacent the glottic opening.

40. The method as set forth in claim 39, which, prior to the step of positioning the guidewire, further comprises the steps of:
positioning a retrograde intubation device, having a hollow passage therethrough, within, and through, the cricoidthyroid membrane, the glottic opening at the vocal cords, the pharyngeal area, the oral cavity and at least to a location outside of the patient's oral cavity opening; and
positioning the guidewire within, and through, the hollow passage of the retrograde intubation device and at least to the location outside of the patient's oral cavity opening.

41. The method as set forth in claim 40 which further comprises the steps of:
moving the carrier device guidingly along the guidewire and the retrograde intubation device from the location outside of the patient's oral cavity opening, through the oral cavity opening, the oral cavity, the pharyngeal area, and at least into the position adjacent the glottic opening.

42. The method as set forth in claim 41, which, prior to moving the carrier device guidingly along the guide wire and the hollow retrograde intubation device, further comprises the steps of:
withdrawing the retrograde intubation device from within the patient.

43. The method as set forth in claim 42, which, after the step of moving the carrier device, further comprises the step of:
withdrawing the guidewire from within the patient.

44. A method of providing an open airway for a patient, where the patient has a neck, a cricoidthyroid membrane, a trachea, lungs, vocal cords with a foreign body lodged therein, a glottic opening at the vocal cords, and a pharyngeal area, which comprises the steps of:
mounting a deflated miniature inflatable cuff on a retrograde device;
positioning the retrograde device within, and through, the cricoidthyroid membrane and into the trachea, with the deflated miniature inflatable cuff being located in the trachea between the vocal cords and the lungs;
inflating the miniature cuff; and
moving the retrograde device toward the lodged foreign body to dislodge the foreign body into the pharyngeal area.

45. A method of providing an open airway for a patient, where the patient has a neck, a cricoidthyroid membrane, a trachea, vocal cords, a glottic opening at the vocal cords, a pharyngeal area, an oral cavity and an oral cavity opening, which comprises the steps of:
forming a carrier device having an outer sleeve, with a hollow passage therethrough, and an inner sleeve, with a hollow passage therethrough, and the inner sleeve being slidably mounted within the hollow passage of the outer sleeve;
positioning a retrograde device within, and through, the cricoidthyroid membrane, the glottic opening at the vocal cords, the pharyngeal area, the oral cavity and at least to a location outside of the patient's oral cavity opening; and
moving the carrier device with the hollow passage of the inner sleeve being guidingly moved along the retrograde device from the location outside of the patient's oral cavity opening, through the oral cavity opening, the oral cavity, the pharyngeal area, and at least into a position adjacent the glottic opening.

46. The method as set forth in claim 45, which further comprises the step of:
withdrawing the outer sleeve from assembly with the inner sleeve, and out of the patient.

47. The method as set forth in claim 45, which, during the forming of the carrier device, further comprises the step:
attaching a foreign-body dislodging means to the inner sleeve.

48. The method as set forth in claim 47, which further comprises the step:
moving the inner sleeve to move the foreign-body dislodging means into dislodging engagement with the foreign body.

49. A method of providing an open airway for a patient, where the patient has a neck, a cricoidthyroid membrane, a trachea, vocal cords, a glottic opening at the vocal cords, a pharyngeal area, an oral cavity and an oral cavity opening, which comprises the steps of:
forming a rod element;
attaching a foreign-body dislodging means to the rod element.
moving the rod element from a location outside of the patient's oral cavity opening, through the oral cavity opening, the oral cavity, the pharyngeal area, and at least into a position adjacent the glottic opening; and
moving the rod element to move the foreign-body dislodging means into dislodging engagement with the foreign body.
